(19) United States
(12) Patent Application Publication
(10) Pub. No.: US 2022/0401350 A1
(43) Pub. Date: Dec. 22, 2022

(54) Personal Care Compositions and Methods for the Same

(71) Applicant: Colgate-Palmolive Company, New York, NY (US)

(72) Inventors: Qiang WU, Hillsborough, NJ (US); Thomas BOYD, Metuchen, NJ (US)

(73) Assignee: Colgate-Palmolive Company, New York, NY (US)

(21) Appl. No.: 17/250,410

(22) PCT Filed: Dec. 4, 2020
(86) PCT No.: PCT/US2020/070849

§ 371 Date: Jan. 19, 2021

Related U.S. Application Data
(60) Provisional application No. 62/948,493, filed on Dec. 16, 2019.

(51) Int. Cl. A61K 8/9789; A61K 8/92; A61K 8/34; A61Q 19/00

(52) U.S. Cl. CPC A61K 8/9789; A61K 8/922; A61K 8/34; A61Q 19/005

(57) ABSTRACT

Personal care compositions and methods for treating or reducing skin irritation or skin inflammation. The personal care composition may include a carrier, one or more plant oils, and a cannabinoid source. The one or more plant oils and the cannabinoid source may each be present in an effective amount to treat or reduce skin irritation or skin inflammation. The method for treating or reducing skin irritation or skin inflammation may include contacting the personal care composition with skin, and optionally, decreasing the amount of IL-1α in or on the skin.

### BACKGROUND

Personal care compositions may often incorporate beneficial components or benefit agents to impart benefits or properties to the compositions. For example, skincare compositions may often incorporate skin soothing agents to provide the composition with the ability to ameliorate, reduce, or treat skin irritation. While the incorporation of these benefit agents has exhibited efficacy in enhancing the respective properties of the personal care compositions, the search for new benefit agents exhibiting improved benefits is ongoing.

What is needed, then, are personal care compositions incorporating improved benefit agents, and methods for the same.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing a personal care composition including a carrier, one or more plant oils, and a cannabinoid source. The one or more plant oils and the cannabinoid source may each be present in an effective amount to treat or reduce skin irritation or skin inflammation.

In at least one example, the one or more plant oils may include one or more of the following: palm kernel oil, coconut oil, avocado oil, canola oil, corn oil, cottonseed oil, olive oil, palm oil, high-oleic sunflower oil, mid-oleic sunflower oil, sunflower oil, palm stearin oil, palm kernel olein oil, safflower oil, babassu oil, sweet almond oil, castor oil, canola oil, soybean oil, olive oil, acai oil, andiroba oil, apricot kernel oil, argan oil, passion fruit oil, manila oil, mango oil, shea oil, macadamia nut oil, brazil nut oil, borage oil, copaiba oil, grape seed oil, buriti oil, sesame oil, flaxseed oil, blueberry oil, cranberry oil, blackberry oil, plum oil, raspberry oil, camelina oil, camellia oil, walnut oil, wheat germ oil, calendula oil, cherry kernel oil, cucumber seed oil, papaya oil, aloe vera oil, or mixtures or combinations thereof.

In at least one example, the one or more plant oils includes flaxseed oil, hemp seed oil, or a combination of flaxseed oil and hemp seed oil.

In at least one example, the one or more plant oils may be present in an amount of from greater than 0 weight % to less than 40 weight %, about 0.0001 weight % to about 10 weight %, or about 0.00025 weight % to about 1 weight %, based on a total weight of the personal care composition.

In at least one example, the cannabinoid source may include one or more cannabinoids, optionally the one or more cannabinoids may include one or more of cannabichromene, cannabichromevarin, cannabigerol, cannabigerovarin, cannabigerol monomethyl ether, cannabielsoin, cannabicitran, cannabidiol, cannabidiolic acid, cannabinol, cannabidivarin, cannabicyclol, cannabivarin, tetrahydrocannabivarin, Δ9-tetrahydrocannabinol, tetrahydrocannabinolic acid, or combinations thereof.

In at least one example, the cannabinoid source may include one or more of the following: cannabichromene, cannabigerol, cannabidiol, cannabinol, or combinations thereof.

In at least one example, the cannabinoid source may be substantially free, preferably, free of a psychoactive cannabinoid. For example, the cannabinoid source may be substantially free, preferably, free of Δ9-tetrahydrocannabinol.

In at least one example, the cannabinoid source includes cannabidiol.

In at least one example, the cannabinoid source may be present in an amount of from greater than 0 weight % to less than 40 weight %, about 0.0001 weight % to about 10 weight %, or about 0.00025 weight % to about 1 weight %, based on a total weight of the personal care composition.

In at least one example, the cannabinoid source may include cannabis sativa seed oil, hemp seed oil, or a combination thereof.

In at least one example, the one or more plant oils and the cannabinoid source may be present in a ratio effective to treat, decrease, reduce, or prevent skin irritation and/or skin inflammation. In at least one example, the one or more plant oils and the cannabinoid source may be present in a ratio of from about 1:0.1 to about 1:3, more preferably from about 1:0.25 to about 1:2.

In at least one example, the carrier is a solid carrier. In another example, the carrier is a liquid carrier.

In at least one example, the personal care composition is a skin care product.

The foregoing and/or other aspects and utilities embodied in the present disclosure may be achieved by providing a method for preparing any one or more of the personal care compositions disclosed herein. The method may include contacting the carrier, the one or more plant oils, and the cannabinoid source with one another.

The foregoing and/or other aspects and utilities embodied in the present disclosure may be achieved by providing a method for treating or reducing skin irritation or skin inflammation. The method may include contacting the skin with any one or more of the personal care compositions disclosed herein. The method may further include decreasing the amount of IL-1αin or on the skin.

In at least one example, the method may further include determining the presence of the skin irritation or the skin inflammation. In one example, determining the presence of the skin irritation or the skin inflammation may include measuring an elevated amount of IL-1α in the skin relative to a population baseline IL-1α value or a previous individual baseline IL-1α value.

The foregoing and/or other aspects and utilities embodied in the present disclosure may be achieved by providing any one or more of the personal care compositions disclosed herein for use in treating or reducing skin irritation or skin inflammation.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that personal care compositions including a synergistic combination of flaxseed oil (FSO) and a cannabinoid source, such as cannabidiol (CBD), significantly decreased the measured amount of IL-1α, a marker for skin inflammation and irritation, in skin. The surprising and unexpected decrease in IL-1α was demonstrated when FSO and CBD were present in a ratio of from about 1:0.25 to about 1:2.

### COMPOSITIONS

Compositions disclosed herein may be or include a personal care product or a personal care composition thereof. For example, compositions disclosed herein may be a personal care composition, a personal care product, or form a portion of the personal care composition or the personal care product. As used herein, the term or expression “personal care composition” may refer to a composition for topical application to skin of mammals, especially humans. The personal care composition may generally be a leave-on personal care composition or rinse off personal care composition, and may include any product applied to a human body. The personal care composition is preferably a leave-on personal care composition. The personal care composition may be in any suitable form. Illustrative forms of the personal care composition may be or include, but is not limited to, a liquid, a lotion, a cream, a foam, a scrub, a gel, a soap bar, a toner, a substance or composition applied with an implement or via a face mask, or the like. Illustrative personal care compositions may be or include, but are not limited to, cleansers, leave-on skin lotions or creams, emulsion, shampoos, conditioners, shower gels, antiperspirants, deodorants, depilatories, lipsticks, foundations, mascara, sunless tanners, sunscreen lotions, body washes, soaps, including bar soaps and liquid soaps (e.g., liquid hand soaps), face washes, moisturizers, serums, spot treatments, cosmetics, or the like.

In an exemplary implementation, the compositions disclosed herein may be or include personal care compositions including a carrier, one or more plant oils, and one or more cannabinoid sources. In one example, the personal care composition may include a single plant oil. In another example, the personal care composition may include at least two plant oils. In yet another example, the personal care composition includes the one or more plant oils and the one or more cannabinoid sources in a synergistic amount or ratio (e.g., concentration, weight, or volume ratio). In at least one implementation, the personal care composition includes a synergistic amount or ratio of flaxseed oil (FSO) and a cannabinoid source. For example, the personal care composition includes a synergistic amount or ratio of flaxseed oil (FSO) and the cannabinoid source, where the cannabinoid source includes cannabidiol (CBD), and where the CBD may be a solution of CBD in hemp seed oil.

The personal care compositions disclosed herein may also be capable of or configured to significantly decrease the measured amount of IL-1α, a marker for skin inflammation and irritation. As such, the personal care composition disclosed herein may be capable of or configured to treat, decrease, reduce, or otherwise prevent skin inflammation and/or irritation. For example, the personal care compositions disclosed herein may be capable of or configured to significantly decrease the measured amount of IL-1α when the plant oil, such as FSO, and the cannabinoid source are present in a synergistic amount or ratio, such as a ratio of from about 4:1 (or about 1:0.25) to about 1:2. Accordingly, the personal care compositions disclosed herein may be utilized in the treatment and/or prevention of skin irritation or skin inflammation.

The personal care composition may include one or more plant oils. For example, the personal care composition may include a single plant oil or a combination of two or more plant oils. As used herein, “plant oil” may refer to a natural oil that is completely obtained from a plant, or a manufactured oil made by blending at least two components of oil (e.g., triglycerides, saturated and/or unsaturated fatty acids, etc.) to substantially mimic the composition of a natural plant oil or provide an oil substantially similar in composition to a plant oil. For example, a manufactured oil substantially similar in composition to a plant oil may include at least 50 weight %, at least 60 weight %, at least 70 weight %, at least 80 weight %, at least 90 weight %, at least 95 weight %, at least 98 weight %, at least 99 weight %, at least 99.5 weight %, at least 99.9 weight %, or 100 weight % of the components that are naturally found in the plant oil that the manufactured oil is designed to substantially mimic.

Illustrative plant oils may be or include, but are not limited to, palm kernel oil, coconut oil, avocado oil, canola oil, corn oil, cottonseed oil, olive oil, palm oil, high-oleic sunflower oil, mid-oleic sunflower oil, sunflower oil, palm stearin oil, palm kernel olein oil, safflower oil, babassu oil, sweet almond oil, castor oil, canola oil, soybean oil, olive oil, acai oil, andiroba oil, apricot kernel oil, argan oil, passion fruit oil, manila oil, mango oil, shea oil, macadamia nut oil, brazil nut oil, borage oil, copaiba oil, grape seed oil, buriti oil, sesame oil, flaxseed oil or linseed oil, blueberry oil, cranberry oil, blackberry oil, plum oil, raspberry oil, camelina oil, camellia oil, walnut oil, wheat germ oil, calendula oil, cherry kernel oil, cucumber seed oil, papaya oil, aloe vera oil, hemp oil, hemp seed oil, or the like, or mixtures or combinations thereof. In at least one particular implementation, the one or more plant oils may include flaxseed oil (FSO). In another implementation, the one or more plant oils may include FSO and hemp seed oil (HSO).

In at least one implementation, any one or more of the plant oils may be present in the personal care composition in an effective amount or a therapeutically effective amount. As used herein, the expression or term “effective amount of one or more plant oils” or the like may refer to an amount of one or more plant oils sufficient to interact or work synergistically with the cannabinoid source to elicit a response (e.g., biological, medical, etc.) of a tissue, system, animal, or human that is being sought. For example a plant oil, such as FSO, may be present in the personal care composition in an effective amount to interact or work synergistically with the cannabinoid source, such as CBD (e.g., CBD in HSO), to reduce skin irritation and/or skin inflammation.

The amount or concentration of any one or more of the plant oils present in the personal care composition may vary widely. In at least one implementation, any one or more of the plant oils may be present in the personal care composition in an amount sufficient to deliver an effective amount and/or ratio, as disclosed herein, of the one or more plant oils to skin cells when applied to an outer surface of the skin or outer dermis. It should be appreciated that the amount or concentration of the one or more plant oils present in the in the personal care composition may be relatively greater than the effective amount, as penetration of the one or more plant oils from the outer dermis to the skin cells may be at least partially determined by varying factors, as is known by those having ordinary skill in the art.

In at least one implementation, the amount of any one or more of the plant oils (e.g., each or a combination) present in the personal care composition may be from greater than 0 weight % to less than or equal to 40 weight %, based on a total weight of the personal care composition. For example, any one or more of the plant oils may be present in the personal care composition in an amount of from greater than 0 weight %, about 0.00025 weight %, about 0.0001 weight %, about 0.001 weight %, about 0.01 weight %, about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, about 1 weight %, about 1.2 weight %, about 1.4 weight %, about 1.6 weight %, about 1.8 weight %, about 2 weight %, about 3 weight %, about 4 weight %, about 5 weight %, about 6 weight %, about 7 weight %, about 8 weight %, about 9 weight %, about 10 weight %, about 15 weight %, or about 20 weight % to about 25 weight %, about 30 weight %, about 35 weight %, about 40 weight %, based on a total weight of the personal care composition. In another example, any one or more of the plant oils may be present in the personal care composition in an amount of at least 0.00001 wt %, at least 0.0001 weight %, at least 0.001 weight %, at least 0.01 weight %, at least 0.1 weight %, at least 1.0 weight %, at least 1.5 weight %, at least 2.0 weight %, at least 2.5 weight %, at least 3.0 weight %, at least 3.5 weight %, at least 4.0 weight %, at least 5 weight %, at least 10 weight %, at least 20 at least %, at least 30 weight %, or more, based on a total weight of the personal care composition. In yet another example, any one or more of the plant oils may be present in the personal care composition in an amount of from greater than 0 weight % to less than 40 weight %, less than 35 weight %, less than 30 weight %, less than 25 weight %, less than 20 weight %, less than 15 weight %, less than 10 weight %, less than 9 weight %, less than 8 weight %, less than 7 weight %, less than 6 weight %, less than 5 weight %, less than 4 weight %, less than 3 weight %, less than 2 weight %, less than 1 weight %, less than 0.1 weight %, less than 0.01 weight %, less than 0.001 weight %, less than 0.0001 weight %, less than 0.00001 weight %, based on a total weight of the personal care composition. In yet another example, any one or more of the plant oils may be present in the personal care composition in an amount of from greater than 0 weight % to about 5 weight %, about 0.01 weight % to about 5 weight %, about 0.1 weight % to about 4.9 weight %, about 0.2 weight % to about 4.8 weight %, about 0.3 weight % to about 4.7 weight %, about 0.4 weight % to about 4.6 weight %, about 0.5 weight % to about 4.5 weight %, about 0.6 weight % to about 4.4 weight %, about 0.7 weight % to about 4.3 weight %, about 0.8 weight % to about 4.2 weight %, about 0.9 weight % to about 4.1 weight %, about 1 weight % to about 4 weight %, about 1.2 weight % to about 3.8 weight %, about 1.4 weight % to about 3.6 weight %, about 1.6 weight % to about 3.4 weight %, about 1.8 weight % to about 3.2 weight %, about 2 weight % to about 3 weight %, about 2.2 weight % to about 2.8 weight %, or about 2.4 weight % to about 2.6 weight %, based on a total weight of the personal care composition.

The personal care composition may include one or more cannabinoid sources. The cannabinoid source may include one or more cannabinoids. Illustrative cannabinoid sources may be or include, but are not limited to, cannabichromene (CBC), cannabichromevarin (CBCV), cannabigerol (CBG), cannabigerovarin (CBGV), cannabigerol monomethyl ether (CBGM), cannabielsoin (CBE), cannabicitran (CBT), cannabidiol (CBD), cannabidiolic acid (CBDA), cannabinol (CBN), cannabidivarin (CBDV), cannabicyclol (CBL), cannabivarin (CBV), tetrahydrocannabivarin (THCV), Δ9-tetrahydrocannabinol (THC), tetrahydrocannabinolic acid (THCA), or the like, or combinations thereof. In at least one implementation, the cannabinoid source may include a cannabinoid selected from CBC, CBG, CBD, CBN, or combinations thereof. In a particular implementation, the cannabinoid source includes at least CBD. For example, the cannabinoid source may include, consists essentially of, or consists of CBD as represented by Formula (1).

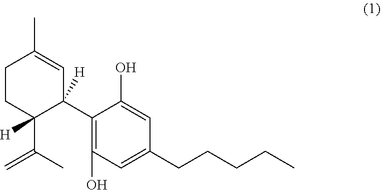 [SMILES: C=C(C)[C@@H]1CCC(C)=C[C@H]1c1c(O)cc(CCCCC)cc1O]

The cannabinoid source may be or include a non-psychoactive cannabinoid. For example, the cannabinoid source may be free or substantially free of any one or more psychoactive cannabinoids, such as Δ9-tetrahydrocannabinol (THC). For example, the cannabinoid source may include less than or equal to about 0.3 weight % THC, less than or equal to about 0.2 weight % THC, less than or equal to about 0.1 weight % THC, less than or equal to about 0.05 weight % THC, or less than or equal to about 0.01 weight % THC, based on a total weight of the composition or the cannabinoid source thereof.

In at least one implementation, the cannabinoid source or the cannabinoids thereof may be present in the form of an oil. For example, the cannabinoid source may be an oil including one or more cannabinoids. In at least one example, the oil including the one or more cannabinoids may be or include a cannabis sativa seed oil, hemp seed oil, or combinations thereof. In at least one implementation, the cannabinoid source may include one or more cannabinoids and HSO or cannabis sativa seed oil (CSO). The HSO or the CSO may be a carrier for the one or more cannabinoids. For example, the one or more cannabinoids may be disposed, mixed, suspended, dispersed, dissolved, combined with, or otherwise contacted with the HSO or the CSO.

In at least one implementation, the cannabinoid source may be present in the personal care composition in an effective amount or a therapeutically effective amount. As used herein, the expression or term “effective amount of the cannabinoid source” or the like may refer to an amount of the cannabinoid source sufficient to interact or work synergistically with the one or more plant oils to elicit a response (e.g., biological, medical, etc.) of a tissue, system, animal, or human that is being sought. For example the cannabinoid source, such as CBD (e.g., CBD in HSO), may be present in the personal care composition in an effective amount to interact or work synergistically with the one or more plant oils, such as FSO, to reduce skin irritation and/or inflammation.

The amount or concentration of any one or more of the cannabinoid sources present in the personal care composition may vary widely. In at least one implementation, any one or more of the cannabinoid sources or the cannabinoids thereof may be present in the personal care composition in an amount sufficient to deliver an effective amount and/or ratio, as disclosed herein, of the cannabinoid source or the cannabinoids thereof to skin cells when applied to an outer surface of the skin or outer dermis. It should be appreciated that the amount or concentration of the cannabinoid source or the cannabinoids thereof present in the in the personal care composition may be relatively greater than the effective amount, as penetration of the cannabinoid source or the cannabinoids thereof from the outer dermis to the skin cells may be at least partially determined by varying factors, as is known by those having ordinary skill in the art.

In at least one implementation, the amount of the cannabinoid source or the one or more cannabinoids thereof (e.g., each or a combination) present in the personal care composition may be from greater than 0 weight % to less than or equal to 40 weight %, based on a total weight of the personal care composition. For example, the cannabinoid source or the one or more cannabinoids thereof may be present in the personal care composition in an amount of from greater than 0 weight %, about 0.0001 weight %, about 0.00025 weight %, about 0.001 weight %, about 0.01 weight %, about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, about 1 weight %, about 1.2 weight %, about 1.4 weight %, about 1.6 weight %, about 1.8 weight %, about 2 weight %, about 3 weight %, about 4 weight %, about 5 weight %, about 6 weight %, about 7 weight %, about 8 weight %, about 9 weight %, about 10 weight %, about 15 weight %, or about 20 weight % to about 25 weight %, about 30 weight %, about 35 weight %, about 40 weight %, based on a total weight of the personal care composition. In another example, the cannabinoid source or the one or more cannabinoids thereof may be present in the personal care composition in an amount of at least 0.00001 wt %, at least 0.0001 weight %, at least 0.001 weight %, at least 0.01 weight %, at least 0.1 weight %, at least 1.0 weight %, at least 1.5 weight %, at least 2.0 weight %, at least 2.5 weight %, at least 3.0 weight %, at least 3.5 weight %, at least 4.0 weight %, at least 5 weight %, at least 10 weight %, at least 20 at least %, at least 30 weight %, or more, based on a total weight of the personal care composition. In yet another example, the cannabinoid source or the one or more cannabinoids thereof may be present in the personal care composition in an amount of from greater than 0 weight % to less than 40 weight %, less than 35 weight %, less than 30 weight %, less than 25 weight %, less than 20 weight %, less than 15 weight %, less than 10 weight %, less than 9 weight %, less than 8 weight %, less than 7 weight %, less than 6 weight %, less than 5 weight %, less than 4 weight %, less than 3 weight %, less than 2 weight %, less than 1 weight %, less than 0.1 weight %, less than 0.01 weight %, less than 0.001 weight %, less than 0.0001 weight %, less than 0.00001 weight %, based on a total weight of the personal care composition. In yet another example, the cannabinoid source or the one or more cannabinoids thereof may be present in the personal care composition in an amount of from greater than 0 weight % to about 5 weight %, about 0.01 weight % to about 5 weight %, about 0.1 weight % to about 4.9 weight %, about 0.2 weight % to about 4.8 weight %, about 0.3 weight % to about 4.7 weight %, about 0.4 weight % to about 4.6 weight %, about 0.5 weight % to about 4.5 weight %, about 0.6 weight % to about 4.4 weight %, about 0.7 weight % to about 4.3 weight %, about 0.8 weight % to about 4.2 weight %, about 0.9 weight % to about 4.1 weight %, about 1 weight % to about 4 weight %, about 1.2 weight % to about 3.8 weight %, about 1.4 weight % to about 3.6 weight %, about 1.6 weight % to about 3.4 weight %, about 1.8 weight % to about 3.2 weight %, about 2 weight % to about 3 weight %, about 2.2 weight % to about 2.8 weight %, or about 2.4 weight % to about 2.6 weight %, based on a total weight of the personal care composition.

As discussed above, the personal care composition may include a synergistic amount and/or combination of the one or more plant oils and the cannabinoid sources, or the one or more cannabinoids thereof. For example, the personal care composition may include a synergistic combination of the one or more plant oils and the cannabinoid source.

In at least one implementation, the one or more plant oils and the cannabinoid source may be present in an effective ratio (i.e., concentration, weight, or volume ratio) or a therapeutically effective ratio (i.e., concentration, weight, or volume ratio) to elicit a response (e.g., biological medical, etc.) of a tissue, system, animal, or human that is being sought. For example, the one or more plant oils, such as FSO, and the cannabinoid source or the one or more cannabinoids thereof, such as CBD, may be present in an effective concentration, weight, or volume ratio or a therapeutically effective concentration, weight, or volume ratio to treat, decrease, reduce, or otherwise prevent skin irritation and/or inflammation.

In at least one implementation, the concentration, weight, or volume ratio of the one or more plant oils to the cannabinoid source or the one or more cannabinoids thereof may be from about 1:0.1 to about 1:5. For example, the concentration, weight, or volume ratio of the plant oil to the cannabinoid source may be about 1:0.1, about 1:0.15, about 1:0.2, about 1:0.25, about 1:0.3, about 1:0.35, about 1:0.4, about 1:0.45, about 1:0.5, about 1:0.55, about 1:0.6, about 1:0.65, about 1:0.7, about 1:0.75, about 1:0.8, about 1:0.85, about 1:0.9, about 1:0.95, about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.1, about 1:2.2, about 1:2.3, about 1:2.4, about 1:2.5, about 1:2.6, about 1:2.7, about 1:2.8, about 1:2.9, about 1:3, about 1:3.1, about 1:3.2, about 1:3.3, about 1:3.4, about 1:3.5, about 1:3.6, about 1:3.7, about 1:3.8, about 1:3.9, about 1:4, about 1:4.1, about 1:4.2, about 1:4.3, about 1:4.4, about 1:4.5, about 1:4.6, about 1:4.7, about 1:4.8, about 1:4.9, or about 1:5. In another example, the concentration, weight, or volume ratio of the plant oil to the cannabinoid source may be from greater than or equal to about 1:0.1 and less than or equal to about 1:5, less than or equal to about 1:4, less than or equal to about 1:3, less than or equal to about 1:2.8, less than or equal to about 1:2.6, less than or equal to about 1:2.4, less than or equal to about 1:2.3, less than or equal to about 1:2.2, less than or equal to about 1:2, less than or equal to about 1:1.8, less than or equal to about 1:1.6, less than or equal to about 1:1.4, less than or equal to about 1:1.2, less than or equal to about 1:1, less than or equal to about 1:0.8, less than or equal to about 1:0.7, less than or equal to about 1:0.6, less than or equal to about 1:0.5, less than or equal to about 1:0.45, less than or equal to about 1:0.4, less than or equal to about 1:0.35, less than or equal to about 1:0.3, less than or equal to about 1:0.25, less than or equal to about 1:0.2, or less than or equal to about 1:0.15. In yet another example, the concentration, weight, or volume ratio of the plant oil to the cannabinoid source may be from greater than or equal to about 1:0.1, greater than or equal to about 1:0.15, greater than or equal to about 1:0.2, greater than or equal to about 1:0.25, greater than or equal to about 1:0.3, greater than or equal to about 1:0.35, greater than or equal to about 1:0.4, a greater than or equal to bout 1:0.45, greater than or equal to about 1:0.5, greater than or equal to about 1:0.55, greater than or equal to about 1:0.6, greater than or equal to about 1:0.65, greater than or equal to about 1:0.7, greater than or equal to about 1:0.75, greater than or equal to about 1:0.8, greater than or equal to about 1:0.85, greater than or equal to about 1:0.9, greater than or equal to about 1:0.95, greater than or equal to about 1:1, greater than or equal to about 1:1.1, greater than or equal to about 1:1.2, greater than or equal to about 1:1.3, greater than or equal to about 1:1.4, greater than or equal to about 1:1.5, greater than or equal to about 1:1.6, greater than or equal to about 1:1.7, greater than or equal to about 1:1.8, greater than or equal to about 1:1.9, greater than or equal to about 1:2, greater than or equal to about 1:2.1, greater than or equal to about 1:2.2, greater than or equal to about 1:2.3, greater than or equal to about 1:2.4, greater than or equal to about 1:2.5, greater than or equal to about 1:2.6, greater than or equal to about 1:2.7, greater than or equal to about 1:2.8, greater than or equal to about 1:2.9, greater than or equal to about 1:3, greater than or equal to about 1:3.1, greater than or equal to about 1:3.2, greater than or equal to about 1:3.3, greater than or equal to about 1:3.4, greater than or equal to about 1:3.5, greater than or equal to about 1:3.6, greater than or equal to about 1:3.7, greater than or equal to about 1:3.8, greater than or equal to about 1:3.9, greater than or equal to about 1:4, greater than or equal to about 1:4.1, greater than or equal to about 1:4.2, greater than or equal to about 1:4.3, greater than or equal to about 1:4.4, greater than or equal to about 1:4.5, greater than or equal to about 1:4.6, greater than or equal to about 1:4.7, greater than or equal to about 1:4.8, or greater than or equal to about 1:4.9 to less than or equal to about 1:5. In a particular implementation, the concentration, weight, or volume ratio of the plant oil, such as FSO, to the cannabinoid source, such as CBD in HSO, may be from about 1:0.1 to about 1:3, more preferably from about 1:0.25 to about 1:2. For example, the concentration, weight, or volume ratio of flaxseed oil to a 1% solution of CBD in HSO may be from about 1:0.1 to about 1:3, more preferably from about 1:0.25 to about 1:2.

The personal care composition may include the active ingredients, mixed with, dissolved in, combined with, or otherwise contacted with the carrier or one or more excipients. For example, the personal care composition may include the one or more plant oils and the cannabinoid source or the one or more cannabinoids thereof mixed with, dissolved in, combined with, or otherwise contacted with the carrier or one or more excipients. In at least one implementation, the carrier may be capable of or configured to store, entrain, or otherwise contain the one or more plant oils and the cannabinoid source or the one or more cannabinoids thereof, and deliver the one or more plant oils and the cannabinoid source or the one or more cannabinoids thereof to one or more tissues, such as skin. It should be appreciated that the components or contents of the carrier and the respective amount of each of the components of the carrier may be at least partially determined by the type or use of the personal care product or the composition thereof. Illustrative personal care products or compositions thereof may include, but are not limited to, cleansers, leave-on skin lotions or creams, emulsion, shampoos, conditioners, shower gels, antiperspirants, deodorants, depilatories, lipsticks, foundations, mascara, sunless tanners, sunscreen lotions, body washes, soaps, including bar soaps and liquid soaps (e.g., liquid hand soaps), face washes, moisturizers, serums, spot treatments, cosmetics, or the like. In a preferred implementation, the personal care product or the composition thereof that includes the one or more plant oils and the cannabinoid source or the one or more cannabinoids thereof are solid cleansing compositions, such as bar soaps.

In at least one implementation, the personal care product or the composition thereof may be a skin care product. Illustrative skin care product may be or include, but are not limited to, a lotion, a cosmetic, a sunscreen, or the like. The carrier of the skin care product may include, but is not limited to, any one or more of surfactants, conditioning agents, moisturizers, sunscreens, UV absorbers, antioxidants, enzymes and/or other proteins, vitamins, antibacterial agents, odor reducing agents, steroids, anti-inflammatory agents, naturally and/or non-naturally occurring humectants, skin lipid fluidizers, occlusive agents, amino acids, physical and/or chemical exfoliants, skin whiteners, anti-aging, antiperspirant actives, or the like, or any combination thereof.

In at least one implementation, the personal care product or the composition thereof may be a personal hand and/or body cleansing composition or a personal hand and/or body conditioning composition. Illustrative personal hand and/or body cleansing or conditioning compositions may include, but are not limited to, liquid soaps, bar soaps, body washes, shower gels, lotions, and the like. In a preferred implementation, the personal hand and/or body cleansing or conditioning composition is a solid personal hand and/or solid body cleansing or conditioning composition, such as bar soap. The carrier for the personal hand and/or body cleansing composition or the personal hand and/or body conditioning composition may include, but is not limited to, any one or more of fragrances, essential oils, emulsifying agents, thickening agents, colorants, surfactants, natural actives, therapeutic actives, stain prevention actives, antimicrobial agents, vitamins, natural extracts, amino acids, enzymes and/or other proteins, abrasives, odor control agents, conditioning agents, moisturizers, humectants, occlusive agents, skin lipid fluidizers, lipophilic actives, hydrophilic materials, pearlizers, opacifying agents, sodium soaps, titanium dioxide, fragrances, or the like, or any mixture or combination thereof, in addition to any one or more of the other carrier components as discussed above.

The carrier may be hydrophilic or hydrophobic. The carrier may be anhydrous. The carrier may be a liquid or a solid at room temperature. The carrier may have a viscosity of from about 2,000 centipoise (cP) to about 100,000 cP. For example, the carrier for a shower gel may have a viscosity of from about 2,000 cP to about 16,000 cP. In another example, the carrier for a lotion may have a viscosity of from about 10,000 cP to about 100,000 cP. Accordingly, it should be appreciated that the viscosity of the carrier may vary and may at least partially depend on the type of personal care composition. In an exemplary implementation, the carrier is a solid at room temperature.

Unless otherwise specifically identified, the ingredients for use in the compositions and formulations of the compositions disclosed herein are preferably cosmetically acceptable ingredients. As used herein, the expression “cosmetically acceptable” may refer to a component or ingredient that is suitable for use in a formulation for topical application to human skin. A cosmetically acceptable excipient, may refer to an excipient that is suitable for external application in the amounts and concentrations contemplated in the formulations of the compositions disclosed herein, and includes for example, excipients which are “Generally Recognized as Safe” (GRAS) by the United States Food and Drug Administration (FDA).

### METHODS

The present disclosure may provide methods for preparing a personal care product or a personal care composition thereof. The method may include mixing, stirring, combining, or otherwise contacting the one or more plant oils and the one or more cannabinoid sources with a carrier. In at least one example, the carrier may be a solid cleansing composition, such as a bar soap.

The present disclosure may further provide methods for treating or reducing one or more skin conditions, such as skin irritation or skin inflammation. Illustrative irritation or inflammatory conditions may be or include, but are not limited to, redness, itchiness, pruritus, pyoderma, dermal rashes, chaffing, or the like, or combinations thereof. The method for treating or reducing one or more skin conditions may include contacting an effective amount of any one or more of the personal care compositions disclosed herein with the skin. The method may also include decreasing the amount of IL-1α in and/or on the skin by contacting the effective amount of any one or more of the personal care compositions disclosed with the skin. The method may also include diagnosing or determining the presence of skin irritation and/or skin inflammation. The method for diagnosing or determining the presence of skin irritation and/or skin inflammation may include measuring an elevated amount of IL-1α relative to a population baseline value or a previous individual baseline value. The elevated amount of IL-1α may be at least 1% greater, at least 2% greater, at least 5% greater, at least 8% greater, at least 10% greater, at least 12% greater, at least 15% greater, at least 20% greater, at least 25% greater, at least 30% greater, at least 40% greater, at least 50% greater, at least 60% greater, at least 70% greater, at least 80% greater, at least 90% greater, at least 100% greater, at least 150% greater, at least 200% greater, at least 250% greater, at least 300% greater, at least 400% greater, or at least 500% greater, than the population baseline value or the previous individual baseline value.

The present disclosure may also provide a personal care composition including a carrier, one or more plant oils, and a cannabinoid source for use in treating one or more skin conditions, such as skin irritation and/or skin inflammation. The one or more plant oils may include FSO. The cannabinoid source may include one or more cannabinoids. The one or more cannabinoids of the cannabinoid source may include CBD. The CBD may be a 1% solution of CBD in HSO.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example

An in vitro study was conducted to determine the efficacy of varying concentrations of flaxseed oil (FSO) and cannabidiol (CBD) in hemp seed oil (HSO) for preventing, reducing, and/or treating skin irritation. Particularly, an in vitro study was conducted on keratinocyte cell skin models to determine the effect or efficacy of varying concentrations of FSO and CBD in HSO in reducing Interleukin 1 alpha (IL-1α). It should be appreciated that IL-1α, or hematopoietin 1, is a cytokine of the interleukin 1 family that is encoded by the IL-1α gene. It should further be appreciated that Interleukin 1 is responsible for the production of inflammation and/or irritation of the skin, and has been widely used as a marker or biomarker to evaluate skin irritation.

Stock solutions of FSO and CBD in HSO were prepared. A 100% FSO and a 1% solution of CBD in HSO were utilized to prepare the stock solutions. The 100% FSO was combined with 100% dimethyl sulfoxide (DMSO) in a ratio of about 1:99 to thereby prepare a solution of FSO in DMSO. Similarly, the 1% solution of CBD in HSO was combined with 100% DMSO in a ratio of about 1:99 to thereby prepare a solution of CBD. 100 μL of each of the solutions of FSO in DMSO and CBD in DMSO was then combined with 900 μL phosphate-buffered saline (PBS) to prepare respective stock solutions of FSO and CBD. A stock 10% DMSO solution was prepared by combining 100 μL of DMSO with 900 μL of PBS.

The stock solutions were utilized to prepare eight separate samples (1)-(8) including varying amounts of FSO, CBD, and growth media. The amounts or concentrations of each of the samples (1)-(8) are summarized in Table 1. A brief description of each of the samples (1)-(8) is summarized in Table 2.

TABLE 1

| | Composition of Samples (1)-(8) | | | | | | | |
|---|---|---|---|---|---|---|---|---|
| | 1 | 2 | 3 | 4 | 5 | 6 | 7 | 8 |
| Ingredient | mL | mL | mL | mL | mL | mL | mL | mL |
| FSO | 0 | 0.1 | 0 | 0 | 0 | 0.1 | 0.1 | 0.1 |
| (0.1% soln) | | | | | | | | |
| CBD | 0 | 0 | 0.2 | 0.1 | 0.025 | 0.2 | 0.1 | 0.025 |
| (0.1% soln) | | | | | | | | |
| Media | 9.8 | 9.9 | 9.8 | 9.9 | 9.975 | 9.7 | 9.8 | 9.875 |
| DMSO | 0.2 | 0 | 0 | 0 | 0 | 0 | 0 | 0 |

TABLE 2
Brief Description of Samples (1)-(8)

| Sample | Brief Description |
|---|---|
| 1 | Vehicle Control |
| 2 | 0.001% FSO |
| 3 | 0.002% CBD |
| 4 | 0.001% CBD |
| 5 | 0.00025% CBD |
| 6 | 0.001% FSO + 0.002% CBD |
| 7 | 0.001% FSO + 0.001% CBD |
| 8 | 0.001% FSO + 0.00025% CBD |

Keratinocyte cell skin models were utilized as the models in the in vitro study, and IL-1αwas monitored with an IL-1α ELISA Kit. To conduct the in vitro study, Keratinocyte cell were grown in a 6-well plate to reach the confluency of about >60%. About 2 mL of each of the samples (1)-(8) was directly applied to respective wells of the well plate and incubated for 24 hours. After incubation, the cells from each of the wells were collected and used as is. The amount of IL-1α produced in skin models treated with the respective samples (1)-(8) is summarized in Table 3. All measurements were done in triplicate, averaged, and normalized to the total protein in each of the keratinocyte cell skin models.

TABLE 3
Amount of IL-1α Measured from
Skin Models Treated with Samples (1)-(8)

| | | Amount of IL-1α | Change from |
|---|---|---|---|
| # | Sample | (Pg/mL) | Control |
| 1 | Control | 12.7 | 0 |
| 2 | 0.001% FSO | 15.2 | 2.5 |
| 3 | 0.002% CBD | 8.9 | −3.8 |
| 4 | 0.001% CBD | 6.8 | −5.9 |
| 5 | 0.00025% CBD | 5.8 | −6.9 |
| 6 | 0.001% FSO + 0.002% CBD | 4.0 | −8.7 |
| 7 | 0.001% FSO + 0.001% CBD | 5.1 | −7.6 |
| 8 | 0.001% FSO + 0.00025% CBD | 5.2 | −7.5 |

Based on the changes relative to control for samples (2) to (5), respectively, it was expected that the amount of IL-1α in samples (6), (7), and (8) would be a decrease of about −1.3 Pg/mL, about −3.4 Pg/mL, and about −4.4 Pg/mL, respectively. It was surprising and unexpectedly discovered, however, that the combination of FSO and CBD resulted in a significant decrease of about −8.7 Pg/mL, −7.6 Pg/mL, and −7.5 Pg/mL, respectively. The results are especially surprising as the presence of FSO alone, as in sample (1), resulted in an increase in the amount of IL-1α relative to the control. Accordingly, an increase relative to the control would be expected as opposed to the observed decrease when combining FSO with CBD. The results suggest that providing a ratio of FSO to CBD of from about 4:1 (about 1:0.25) to about 1:2 surprisingly and unexpectedly decreased the production of IL-1α in skin. As such, providing a ratio of FSO to CBD of from about 4:1 (about 1:0.25) to about 1:2 would surprisingly and unexpectedly decrease, treat, and/or reduce skin irritation.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

1. A personal care composition, comprising a carrier, one or more plant oils, and a cannabinoid source, wherein the one or more plant oils and the cannabinoid source are each present in an effective amount to treat or reduce skin irritation or skin inflammation.

2. The personal care composition of claim 1, wherein the one or more plant oils comprise one or more of palm kernel oil, coconut oil, avocado oil, canola oil, corn oil, cottonseed oil, olive oil, palm oil, high-oleic sunflower oil, mid-oleic sunflower oil, sunflower oil, palm stearin oil, palm kernel olein oil, safflower oil, babassu oil, sweet almond oil, castor oil, canola oil, soybean oil, olive oil, acai oil, andiroba oil, apricot kernel oil, argan oil, passion fruit oil, manila oil, mango oil, shea oil, macadamia nut oil, brazil nut oil, borage oil, copaiba oil, grape seed oil, buriti oil, sesame oil, flaxseed oil, blueberry oil, cranberry oil, blackberry oil, plum oil, raspberry oil, camelina oil, camellia oil, walnut oil, wheat germ oil, calendula oil, cherry kernel oil, cucumber seed oil, papaya oil, aloe vera oil, or mixtures or combinations thereof.

3. The personal care composition of claim 1, wherein the one or more plant oils comprise flaxseed oil, hemp seed oil, or a combination of flaxseed oil and hemp seed oil.

4. The personal care composition of claim 1, wherein the one or more plant oils are present in an amount of from greater than 0 weight % to less than 40 weight %, about 0.0001 weight % to about 10 weight %, or about 0.00025 weight % to about 1 weight %, based on a total weight of the personal care composition.

5. The personal care composition of claim 1, wherein the cannabinoid source comprises one or more cannabinoids, optionally the one or more cannabinoids comprises one or more of cannabichromene, cannabichromevarin, cannabigerol, cannabigerovarin, cannabigerol monomethyl ether, cannabielsoin, cannabicitran, cannabidiol, cannabidiolic acid, cannabinol, cannabidivarin, cannabicyclol, cannabivarin, tetrahydrocannabivarin, Δ9-tetrahydrocannabinol, tetrahydrocannabinolic acid, or combinations thereof.

6. The personal care composition of claim 1, wherein the cannabinoid source comprises one or more of cannabichromene, cannabigerol, cannabidiol, cannabinol, or combinations thereof.

7. The personal care composition of claim 1, wherein the cannabinoid source is substantially free, preferably, free of a psychoactive cannabinoid.

8. The personal care composition of claim 1, wherein the cannabinoid source is substantially free, preferably, free of Δ9-tetrahydrocannabinol.

9. The personal care composition of claim 1, wherein the cannabinoid source comprises cannabidiol.

10. The personal care composition of claim 1, wherein the cannabinoid source is present in an amount of from greater than 0 weight % to less than 40 weight %, about 0.0001 weight % to about 10 weight %, or about 0.00025 weight % to about 1 weight %, based on a total weight of the personal care composition.

11. The personal care composition of claim 1, wherein the cannabinoid source comprises cannabis sativa seed oil, hemp seed oil, or a combination thereof.

12. The personal care composition of claim 1, wherein the one or more plant oils and the cannabinoid source are present in a ratio effective to treat, decrease, reduce, or prevent skin irritation and/or skin inflammation.

13. The personal care composition of claim 1, wherein the one or more plant oils and the cannabinoid source are present in a ratio of from about 1:0.1 to about 1:3, more preferably from about 1:0.25 to about 1:2.

14. The personal care composition of claim 1, wherein the carrier is a solid carrier.

15. The personal care composition of claim 1, wherein the carrier is a liquid carrier.

16. The personal care composition of claim 1, wherein the personal care composition is a skin care product.

17. A method for preparing the personal care composition of claim 1, the method comprising contacting the carrier, the one or more plant oils, and the cannabinoid source with one another.

18. A method for treating or reducing skin irritation or skin inflammation, the method comprising contacting the skin with the personal care composition of claim 1, optionally, the method further comprising decreasing the amount of IL-1α in or on the skin.

19. The method of claim 18, further comprising determining the presence of the skin irritation or the skin inflammation, optionally, wherein determining the presence of the skin irritation or the skin inflammation comprises measuring an elevated amount of IL-1α in the skin relative to a population baseline IL-1α value or a previous individual baseline IL-1α value.

20. The personal care composition of claim 1 for use in treating or reducing skin irritation or skin inflammation.
